(19) United States
(12) Patent Application Publication
(10) Pub. No.: US 2025/0169850 A1
(43) Pub. Date: May 29, 2025

(54) BATTERY POWERED ROTATIONAL ATHERECTOMY DEVICES

(71) Applicant: Bard Peripheral Vascular, Inc., Franklin Lakes, NJ (US)

(72) Inventor: Peng ZHENG, Chandler, AZ (US)

(73) Assignee: Bard Peripheral Vascular, Inc., Franklin Lakes, NJ (US)

(21) Appl. No.: 18/868,659

(22) PCT Filed: Jun. 21, 2022
(86) PCT No.: PCT/US2022/034210

§ 371 Date: Nov. 22, 2024

(51) Int. Cl. A61B 17/3207; A61B 17/00

(52) U.S. Cl. CPC A61B 17/320758; A61B 2017/00123; A61B 2017/00734

(57) ABSTRACT

A rotational atherectomy device includes an elongate flexible shaft and an abrasive element connected to a distal end of the elongate flexible shaft. A handle includes a housing that includes an electric motor operatively connected to the elongate flexible shaft to rotate the elongate flexible shaft. A power source is electrically connected to the electric motor for supplying power thereto. A controller is configured to monitor a motor supply current of the electric motor and monitor a speed of the electric motor using a signal from a sensor. If the motor supply current is above a first predetermined threshold, the controller reduces a speed of the electric motor while attempting to maintain motor supply current of the electric motor. Then, if the motor supply current is above a second predetermined threshold higher than the first predetermined threshold, the controller stops the electric motor.

### TECHNICAL FIELD

The present invention relates to an atherectomy system, and, more particularly, to an orbital atherectomy system having an abrasive element.

### BACKGROUND

Coronary heart disease may be caused, for example, by atherosclerosis. Atherosclerosis occurs when fat, cholesterol, and/or other substances build up in the walls of blood vessels, forming hard structures called occlusions, e.g., plaques and/or atherosclerotic (stenotic) lesions. Over time, these occlusions may increase in size such that the blood vessels are substantially clogged and/or completely blocked, so as to form a total chronic occlusion (CTO).

Rotational atherectomy is a technique used to abrade, for example, calcified arterial lesions. Rotational atherectomy devices and rotational atherectomy procedures may also be referred to as rotational angioplasty devices and/or rotational angioplasty procedures. One type of rotational atherectomy device is known as an orbital atherectomy device, such as the Diamondback 360® Peripheral Orbital Atherectomy Device available from Cardiovascular Systems Inc. (“CSI”).

Rotational atherectomy devices may include an abrasive element attached to a rotatable elongate flexible driveshaft. The abrasive element may be referred to as a burr, crown, and/or bead. The rotatable elongate flexible driveshaft may be delivered over a guidewire and/or through a sheath to a desired location. The driveshaft may be rotated at high speeds (e.g., between 20,000-160,000 rpm). As the abrasive element rotates, it may be advanced over a stenotic lesion such that the abrasive element contacts the occluding-plaque. In this way, the abrasive element engages the diseased lesion surface and abrades the plaque into very small particles. These small particles may be absorbed by the body or captured via the use of an embolic protection device.

What is need are atherectomy devices that include controllers with improved motor control logic that stop or slow down the motors at high torque events depending on the magnitude of the torque detected by the controllers in order to reduce high torque events and motor stalling conditions.

### SUMMARY

In an embodiment, a rotational atherectomy device includes an elongate flexible shaft and an abrasive element connected to a distal end of the elongate flexible shaft. A handle includes a housing that includes an electric motor operatively connected to the elongate flexible shaft to rotate the elongate flexible shaft. A power source is electrically connected to the electric motor for supplying power thereto. A controller controls operation of the electric motor using the power source. A sensor provides a signal indicative of an operating condition of the electric motor. The controller is configured to monitor a motor supply current of the electric motor and monitor a speed of the electric motor using the signal from the sensor. If the motor supply current is above a first predetermined threshold, the controller reduces a speed of the electric motor while attempting to maintain motor supply current of the electric motor. Then, if the motor supply current is above a second predetermined threshold higher than the first predetermined threshold, the controller stops the electric motor.

In another embodiment, a rotational atherectomy device includes an elongate flexible shaft and an abrasive element connected to a distal end of the elongate flexible shaft. A handle includes a housing. An electric motor is operatively connected to the elongate flexible shaft to rotate the elongate flexible shaft. A power source is electrically connected to the electric motor for supplying power thereto. A controller controls operation of the electric motor using the power source. A sensor provides a signal indicative of an operating condition of the electric motor. The controller is configured to monitor a motor supply current of the electric motor and monitor a speed of the electric motor using the signal from the sensor. If the motor supply current is above a predetermined threshold, a speed of the electric motor and the motor supply current is reduced. Upon a preselected period of time after reducing the speed of the electric motor and the motor supply current, the controller increases the speed of the electric motor and the motor supply current.

### BRIEF DESCRIPTION OF DRAWINGS

The above-mentioned and other features and advantages described herein, and the manner of attaining them, will become more apparent and the disclosure will be better understood by reference to the following description of embodiments taken in conjunction with the accompanying drawings, wherein:

FIG. 1 is a schematic perspective view of an embodiment of a rotational atherectomy device having a handle assembly coupled to a rotational device having an elongate flexible driveshaft and an abrasive element, according to one or more aspects shown and described herein;

FIG. 2 is a schematic side view of the abrasive element of FIG. 1, according to one or more embodiments shown and described herein;

FIG. 3 shows a block diagram of the atherectomy device of FIG. 1 including the handle assembly, flexible driveshaft and abrasive element, according to one or more embodiments shown and described herein;

FIG. 4 shows a control system for use with the atherectomy device of FIG. 1, according to one or more embodiments shown and described herein; and

FIG. 5 shows a control profile for the control system of FIG. 4 based on torque, according to one or more embodiments shown and described herein.

Corresponding reference characters indicate corresponding parts throughout the several views. The exemplifications set out herein illustrate embodiments of the disclosure, and such exemplifications are not to be construed as limiting the scope of the embodiments in any manner.

### DESCRIPTION OF EMBODIMENTS

Embodiments described herein are generally directed to rotational atherectomy devices that include an elongate flexible shaft, an abrasive element connected to a distal end of the elongate flexible shaft and a handle assembly that includes a housing. The handle includes an electric motor operatively connected to the elongate flexible shaft to rotate the elongate flexible shaft. A power source is electrically connected to the electric motor for supplying power thereto. A controller controls operation of the electric motor using the power source. A sensor provides a signal indicative of an operating condition of the electric motor. The controller is configured to monitor a motor supply current of the electric motor and monitor a speed of the electric motor using the signal from the sensor. If the motor supply current is above a first predetermined threshold, a speed of the electric motor is reduced while attempting to maintain motor supply current of the electric motor. If the motor supply current is above a second predetermined threshold higher than the first predetermined threshold, the controller stops the electric motor.

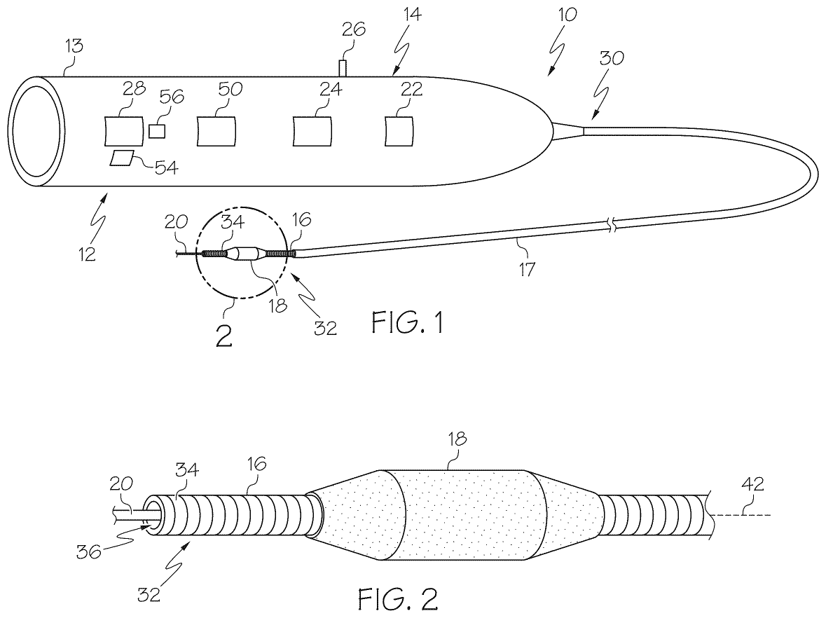

Referring now to the drawings, and more particularly to FIGS. 1 and 2, there is shown a rotational atherectomy system 10 including an atherectomy device 14 that includes a handle assembly 12 with a housing 13 that is configured to rotate and elongate flexible driveshaft 16 at high speeds (e.g., 1000-50,000 rpm). Atherectomy device 14 includes the elongate flexible driveshaft 16 and an abrasive element 18. Elongate flexible driveshaft 16 is flexible so that it may be adaptable to the curvature of the vasculature and extends through a flexible outer conduit 17 (e.g., a catheter). Abrasive element 18 is disposed on and fixedly (i.e., non-detachably or non-removably) attached to elongate flexible driveshaft 16. In the present embodiment, atherectomy device 14 may axially traverse, and rotate around, a guidewire 20. In some embodiments, a lubricant or saline may be supplied through the elongate flexible driveshaft 16 to cool/lubricate the coil shaft/guidewire interface. Abrasive element 18 performs abrasion (e.g., sanding) of an occlusion, e.g., calcified plaques and/or atherosclerotic (stenotic) lesions in the blood vessel, by high speed rotation of atherectomy device 14 with abrasive element 18 in contact with the occlusion, so as to reduce at least a portion of the occlusion into small particles. When abrasive element 18 is spun at high speeds, abrasive element 18 undergoes a concentric motion pathway. In some embodiments, the motion of the rotating abrasive element 18 may be orbital depending on the shape of the abrasive element 18. As examples, symmetric abrasive elements about the rotational axis may be considered rotational and non-symmetric abrasive elements about the rotational axis may be considered orbital.

Handle assembly 12 may include a motor 22, such as a direct current (DC) brushed or brushless motor, a controller 24, and a user interface 26. An on-board battery power supply 28 is connected in electrical communication with motor 22, controller 24, and user interface 26. The motor 22 may operate in a speed range of from about 1000 rpm to 50,000 rpm with a power supply voltage in the range from 5V to 24V. The supply current to the motor 22 may be in a range from 200 mA to 8 A. Electrical power may be supplied to motor 22, controller 24, and user interface 26 via on-board battery power supply 28. In the present embodiment, on-board battery power supply 28 includes a rechargeable or replaceable battery. It may be possible to reduce a number of batteries needed if the drive motor supply voltage is in the range from 8V to 15V and the motor supply current in the range from 1 A to 6 A. Alternatively, an off-board power source, such as an alternating current (AC) wall outlet, may supply electrical power to handle assembly 12.

User interface 26 may include, for example, one or more switches that provide input commands to controller 24. Such user input commands may include, for example, selectable rotational speed commands, motor acceleration and/or torque profile commands, and/or rotational direction commands.

Atherectomy device 14, including elongate flexible driveshaft 16 and abrasive element 18, is configured to extend into a blood vessel, e.g., an artery, of a patient. In the present embodiment, elongate flexible driveshaft 16 may be, for example, an elongate tightly wound metallic coil. Alternatively, elongate flexible driveshaft 16 may be a flexible metal or polymer tube. Elongate flexible driveshaft 16 includes a proximal end portion 30, a distal end portion 32, a distal end 34, and an elongate lumen 36 (FIG. 2), e.g., an elongate guidewire lumen. Distal end 34 is the distal terminus of distal end portion 32 of elongate flexible driveshaft 16.

The proximal end portion 30 of elongate flexible driveshaft 16 is drivably coupled, i.e., connected, e.g., directly or indirectly through a gear train, to a rotatable motor shaft of motor 22. In the present embodiment, distal end portion 32 may constitute, for example, 0.5 to 10 percent of the total length of elongate flexible driveshaft 16. Distal end portion 40 of elongate flexible driveshaft 16 defines a rotational axis 42 about which abrasive element 18 is rotated in unison with elongate flexible driveshaft 16.

Elongate lumen 36 is configured, e.g., in size and shape, to accommodate guidewire 20. Elongate lumen 36 is configured to slidably receive guidewire 20, such that elongate flexible driveshaft 16 that carries abrasive element 18 may be axially advanced over, and rotated around, guidewire 20 and rotational axis 42 In the present embodiment, distal end portion 40 of elongate flexible driveshaft 16 and abrasive element 18 may be longitudinally advanceable, using the conduit 17, over guidewire 20 and into the blood vessel of the patient so as to extend longitudinally outward from the conduit 17 and engage an occlusion in the blood vessel.

The atherectomy device 14 further includes an irrigation pump, represented by element 50, that is located in the handle assembly 12. Locating the irrigation pump 50 in the handle assembly 12 can allow the atherectomy device 14 to be a self-contained, disposable device. The user interface 26 can be provided that can be used to turn the irrigation pump 50 on and off. The irrigation pump 50 can pump a fluid, such as saline, through the flexible conduit 17 and to the distal end 34 of the flexible driveshaft 16 for cooling and lubrication purposes. In some embodiments, the atherectomy device 14 may be a single use device that includes a fluid source 54 inside the handle assembly 12. While the fluid source 54 is shown outside the pump 50, it may be part of the pump 50. The flow rate of the fluid may be from five ml/min to 100 ml/min. The irrigation pump 50 may include an encoder that monitors if the irrigation pump 50 is running at the desired speed and direction during operation. The irrigation pump 50 may be any suitable type of electrical pump, such as an electromechanical pump, a displacement pump, a diaphragm pump, etc.

The controller 24 may provide electrical power from the power supply 28 to a motor 56 of the electrical pump 50 that drives the electrical pump 50. The controller 24 may operate the electrical pump 50 based on input from the user interface 26. The controller 24 may also control operation of the motor 56 of the electrical pump 50 and the motor 22 of the flexible driveshaft 16 based on current operating conditions which is described in greater detail below.

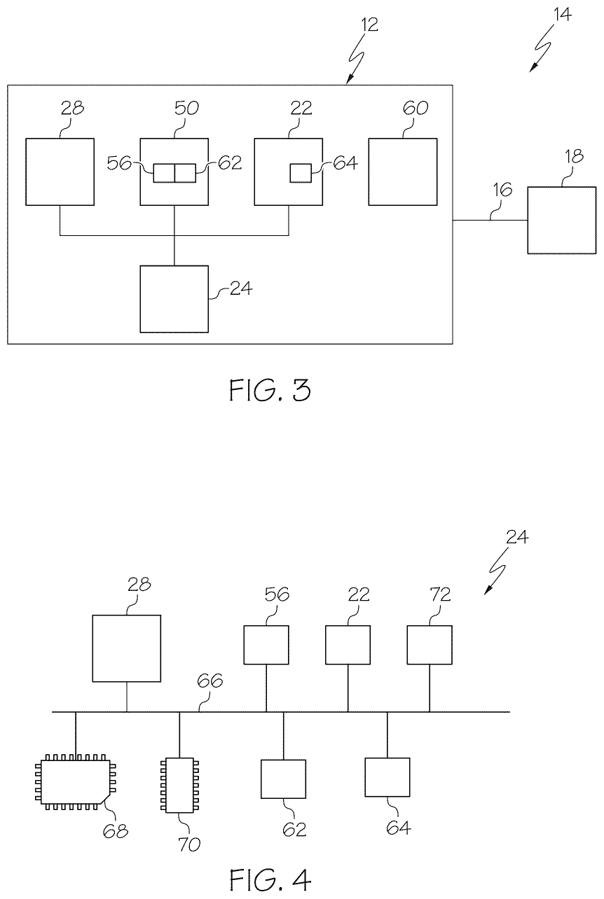

FIG. 3 shows a block diagram of the atherectomy device 14 including the handle assembly 12, flexible driveshaft 16 and abrasive element 18. Located in the handle assembly 12 are the motor 22 of the flexible driveshaft 16, the gear train 60 that provides a mechanical advantage and changes the speed and torque of the flexible driveshaft 16 relative to the motor 22, the electrical pump 50 with motor 56 and the controller 24 that is electrically connected to one or both of the motors 22 and 56. The controller 24 is electrically connected to the power supply 28 for providing power to the motors 22 and 56 and controlling their operation.

The controller 24 may be embodied as a single printed circuit board (PCB) that is configured to monitor the power supply 28 and both of the motors 22 and 56. The output voltage from the power supply 28 may change as the power supply 28 is being used or as current from the power supply 28 increases. The controller 24 monitors the supply voltage determine if the supply voltage drops below a predetermined threshold for the motor 22. The controller 24 also monitors sensors 62 and 64 (encoders) of the motors 22 and 56 and shuts down the system 10 if the motors 22 and 56 do not operate at a desired speed or within a range of speeds.

Referring to FIG. 4, the controller 24 includes a communication path 66, a processor 68 and a memory module 70 that are electrically connected to the power source 28 and the motors 22 and 56 for supplying power and controlling operation of the motors 22 and 56. The memory module 70 includes logic that, when processed by the processor, causes the controller 24 to monitor a motor supply current to the motor 22 and monitor a speed of the motor 22 using the signal from the sensor 64. If the motor supply current is above a first predetermined threshold, the controller 24 reduces a speed of the motor 22 by reducing the supply voltage while attempting to maintain motor supply current of the motor 22. Then, if the supply current goes above a second predetermined threshold higher than the first predetermined threshold, the controller 24 stops the motor 22.

In some embodiments during a stall control mode, the controller 24 may also, if the motor supply current is above a predetermined threshold, reduce a speed of the motor 22 and the motor supply current. The stall control mode may, for example, be activated by the user or the controller 24 using the logic under certain preselected potential stall conditions. Upon a preselected period of time after reducing the speed of the motor 22 and the motor supply current during a stall preventing control operation, increase speed of the motor 22 and the motor supply current to avoid a motor stalling condition.

The controller 24 may also use signals from a sensor 72 (e.g., a contact sensor, proximity sensor, etc.) to detect presence of the guidewire 20, which can be used to detect a guidewire break condition. The user interface 26 may be used to allow motor 22 and 56 speed selection and ON/OFF commands from a switch (e.g., a membrane switch) and control LED lights 74 that provide an indication of operating conditions.

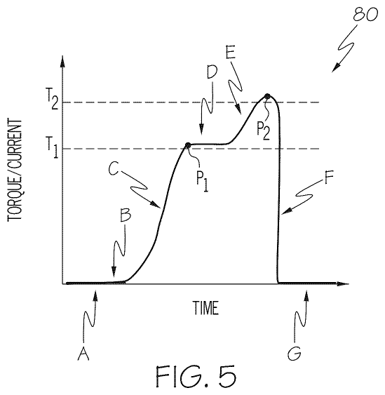

Referring to FIG. 5, a control profile 80 for the controller 24 is illustrated based on torque. At region A, the flexible driveshaft 16 spins freely within a blood vessel with low torque until the abrasive element 18 contacts an occlusion at B. When the abrasive element contacts the occlusion, the rotational speed of the flexible driveshaft 16 slows and the motor torque increases in region C. If the torque increases to at or above a first torque threshold T₁ to P₁ the speed of the motor 22 is reduced while the controller 24 attempts to maintain the torque by maintaining the supply current to the motor 22 in region D. However, if the torque continues to rise to point P₂ above the threshold T₂ in region E, the controller 24 cuts power to the motor 22 and the torque (and speed) go to zero in region F. The controller 24 maintains an OFF state of the motor 22 in region G.

The above-described atherectomy systems provide relatively low-cost atherectomy devices that can be single-use (i.e., disposable) that are self-contained in that they do not need connected to any external devices, such as a power source or fluid line to operate. Thus, little to no setup for the atherectomy devices needs to be done for the atherectomy devices to operate as intended. Further, the atherectomy devices described herein include controllers with improved motor control logic that is used by the controllers to monitor torque at multiple thresholds and first, attempt to maintain torque at a first threshold while decreasing motor speed and, if that does not work and the torque continues to rise to a second predetermined threshold greater than the first predetermined threshold, stop the motors. Thus, the controllers stop or slow down the motors at high torque events depending on the magnitude of the torque detected by the controllers. The atherectomy devices may further be provided with stall control logic that is used to lower the rotational speed at a predetermined torque and then raise the speed and current in order to break a potential stall condition.

Embodiments can be described with reference to the following numbered clauses, with certain features laid out in the dependent clauses:
Clause 1: A rotational atherectomy device comprising: an elongate flexible shaft; an abrasive element connected to a distal end of the elongate flexible shaft; a handle comprising a housing, the handle comprising: an electric motor operatively connected to the elongate flexible shaft to rotate the elongate flexible shaft; a power source electrically connected to the electric motor for supplying power thereto; a controller that controls operation of the electric motor using the power source; and a sensor that provides a signal indicative of an operating condition of the electric motor; wherein the controller is configured to: monitor a motor supply current of the electric motor; monitor a speed of the electric motor using the signal from the sensor; if the motor supply current is above a first predetermined threshold, reduce a speed of the electric motor while attempting to maintain motor supply current of the electric motor; then if the motor supply current is above a second predetermined threshold higher than the first predetermined threshold, stop the electric motor.
Clause 2: The rotational atherectomy device of clause 1 further comprising an irrigation pump electrically connected to the power source.
Clause 3: The rotational atherectomy device of clause 2, wherein the controller controls operation of the irrigation pump.
Clause 4: The rotational atherectomy device of clause 3, wherein the controller monitors a speed of the irrigation pump and shuts down the irrigation pump if the irrigation pump is operating at below a predetermined speed.
Clause 5: The rotational atherectomy device of any of clauses 2-4, wherein the controller comprises a single printed circuit board that controls operation of both the irrigation pump and the electric motor.
Clause 6: The rotational atherectomy device of any of clauses 1-5, wherein the power source is between 5V and 24V.
Clause 7: The rotational atherectomy device of clause 6, wherein the power source comprises one or more batteries located in the handle.
Clause 8: The rotational atherectomy device of any of clauses 1-7, wherein the controller is configured to monitor a motor supply voltage.
Clause 9: The rotational atherectomy device of any of clauses 1-8, wherein the controller is configured to detect presence of a guide wire using input from a sensor.
Clause 10: The rotational atherectomy device of any of clauses 1-9, wherein the handle comprises a user input device, the controller receiving an input from the user input device to control operation of the electric motor.
Clause 11: A rotational atherectomy device comprising: an elongate flexible shaft; an abrasive element connected to a distal end of the elongate flexible shaft; a handle comprising a housing, the handle comprising: an electric motor operatively connected to the elongate flexible shaft to rotate the elongate flexible shaft; a power source electrically connected to the electric motor for supplying power thereto; a controller that controls operation of the electric motor using the power source; and a sensor that provides a signal indicative of an operating condition of the electric motor; wherein the controller is configured to: monitor a motor supply current of the electric motor; monitor a speed of the electric motor using the signal from the sensor; if the motor supply current is above a predetermined threshold, reduce a speed of the electric motor and the motor supply current; upon a preselected period of time after reducing the speed of the electric motor and the motor supply current, increase speed of the electric motor and the motor supply current.
Clause 12: The rotational atherectomy device of clause 11 further comprising an irrigation pump electrically connected to the power source.
Clause 13: The rotational atherectomy device of clause 12, wherein the controller controls operation of the irrigation pump.
Clause 14: The rotational atherectomy device of clause 13, wherein the controller monitors a speed of the irrigation pump and shuts down the irrigation pump if the irrigation pump is operating at below a predetermined speed.
Clause 15: The rotational atherectomy device of any of clauses 12-14, wherein the controller comprises a single printed circuit board that controls operation of both the irrigation pump and the electric motor.
Clause 16: The rotational atherectomy device of any of clauses 11-15, wherein the power source is between 5V and 24V.
Clause 17: The rotational atherectomy device of any of clauses 11-16, wherein the power source comprises one or more batteries located in the handle.
Clause 18: The rotational atherectomy device of any of clauses 11-17, wherein the controller is configured to monitor a motor supply voltage.
Clause 19: The rotational atherectomy device of any of clauses 11-18, wherein the controller is configured to detect presence of a guide wire using input from a sensor.
Clause 20: The rotational atherectomy device of any of clauses 11-19, wherein the handle comprises a user input device, the controller receiving an input from the user input device to control operation of the electric motor.

As used herein, and unless stated otherwise or supplemental to in the context of its use, the term “substantially”, “about”, and other words of degree are relative modifiers intended to indicate permissible variation from the characteristic so modified, possessing more of the physical or functional characteristic than its opposite, and approaching or approximating such a physical or functional characteristic.

While this disclosure has been described with respect to at least one embodiment, the present disclosure can be further modified within the spirit and scope of this disclosure. This application is therefore intended to cover any variations, uses, or adaptations of the embodiments described herein using its general principles. Further, this application is intended to cover such departures from the present disclosure as come within known or customary practice in the art to which this invention pertains and which fall within the limits of the appended claims.

1. A rotational atherectomy device comprising:
an elongate flexible shaft;
an abrasive element connected to a distal end of the elongate flexible shaft;
a handle comprising a housing, the handle comprising:
an electric motor operatively connected to the elongate flexible shaft to rotate the elongate flexible shaft;
a power source electrically connected to the electric motor for supplying power thereto;
a controller that controls operation of the electric motor using the power source; and
a sensor that provides a signal indicative of an operating condition of the electric motor;
wherein the controller is configured to:
monitor a motor supply current of the electric motor;
monitor a speed of the electric motor using the signal from the sensor;
if the motor supply current is above a first predetermined threshold, reduce a speed of the electric motor while attempting to maintain motor supply current of the electric motor; then
if the motor supply current is above a second predetermined threshold higher than the first predetermined threshold, stop the electric motor.

2. The rotational atherectomy device of claim 1 further comprising an irrigation pump electrically connected to the power source that is located in the handle.

3. The rotational atherectomy device of claim 2, wherein the controller controls operation of the irrigation pump.

4. The rotational atherectomy device of claim 3, wherein the controller monitors a speed of the irrigation pump and shuts down the irrigation pump if the irrigation pump is operating at below a predetermined speed.

5. The rotational atherectomy device of claim 4, wherein the controller comprises a single printed circuit board that controls operation of both the irrigation pump and the electric motor.

6. The rotational atherectomy device of claim 1, wherein the power source is between 5V and 24V.

7. The rotational atherectomy device of claim 6, wherein the power source comprises one or more batteries located in the handle.

8. The rotational atherectomy device of claim 1, wherein the controller is configured to monitor a motor supply voltage.

9. The rotational atherectomy device of claim 1, wherein the controller is configured to detect presence of a guide wire using input from another sensor.

10. The rotational atherectomy device of claim 1, wherein the handle comprises a user input device, the controller receiving an input from the user input device to control operation of the electric motor.

11. A rotational atherectomy device comprising:
an elongate flexible shaft;
an abrasive element connected to a distal end of the elongate flexible shaft;
a handle comprising a housing, the handle comprising:
an electric motor operatively connected to the elongate flexible shaft to rotate the elongate flexible shaft;
a power source electrically connected to the electric motor for supplying power thereto;
a controller that controls operation of the electric motor using the power source; and
a sensor that provides a signal indicative of an operating condition of the electric motor;
wherein the controller is configured to:
monitor a motor supply current of the electric motor;
monitor a speed of the electric motor using the signal from the sensor;
if the motor supply current is above a predetermined threshold, reduce a speed of the electric motor and the motor supply current:
upon a preselected period of time after reducing the speed of the electric motor and the motor supply current, increase speed of the electric motor and the motor supply current.

12. The rotational atherectomy device of claim 11 further comprising an irrigation pump electrically connected to the power source.

13. The rotational atherectomy device of claim 12, wherein the controller controls operation of the irrigation pump.

14. The rotational atherectomy device of claim 13, wherein the controller monitors a speed of the irrigation pump and shuts down the irrigation pump if the irrigation pump is operating at below a predetermined speed.

15. The rotational atherectomy device of claim 14, wherein the controller comprises a single printed circuit board that controls operation of both the irrigation pump and the electric motor.

16. The rotational atherectomy device of claim 11, wherein the power source is between 5V and 24V.

17. The rotational atherectomy device of claim 16, wherein the power source comprises one or more batteries located in the handle.

18. The rotational atherectomy device of claim 11, wherein the controller is configured to monitor a motor supply voltage.

19. The rotational atherectomy device of claim 11, wherein the controller is configured to detect presence of a guide wire using input from another sensor.

20. The rotational atherectomy device of claim 11, wherein the handle comprises a user input device, the controller receiving an input from the user input device to control operation of the electric motor.
